(19) United States
(12) Patent Application Publication
(10) Pub. No.: US 2005/0118259 A1
(43) Pub. Date: Jun. 2, 2005

(54) Formulations of quinapril and related ace nhibitors

(76) Inventor: Renir Eyjolfsson, Hafnarfjordur (IS)

Correspondence Address: WENDEROTH, LIND & PONACK, L.L.P., 2033 K STREET N. W., SUITE 800, WASHINGTON, DC, 20006-1021, US

(21) Appl. No.: 10/501,454

(22) PCT Filed: Jan. 14, 2003
(86) PCT No.: PCT/IS03/00002

(30) Foreign Application Priority Data
Jan. 15, 2002 (IS) 6233

(51) Int. Cl. A61K 31/44; A61K 31/405; A61K 31/198; A61K 9/20

(52) U.S. Cl. 424/464; 514/563; 514/357; 514/411; 514/419

(57) ABSTRACT

Stable formulations of ACE-inhibitors compounds such as quinapril can be produced with the use of excipients comprising a basic compound, preferably an alkali-oralkaline earth metal carbonate, and an insoluble alkaline-earth metal carbonate, and an insoluble alkaline-earth metal hydrogen phosphate. Tablets of such formulations have good storage stability, dissolution characteristics, and the formulations are suitable for use in drug combinations.

### FIELD OF THE INVENTION

The present invention is within the field of pharmaceutical formulations of ACE (Angiotensin Converting Enzyme) inhibitors, specifically formulations of quinapril and structurally related compounds.

### TECHNICAL BACKGROUND AND PRIOR ART

Many of the compounds useful as ACE (Angiotensin Converting Enzyme) inhibitors such as as quinapril and structurally related compounds, are prone to degradation. Specifically, such compounds can degrade via (i) cyclization via internal nucleophilic reaction to form substituted diketopiperazines, (ii) hydrolysis of the side-chain ester group, and (iii) oxidation to form products having often unwanted coloration.

Certain stabilizing compositions and formulations of such compounds have been suggested and utilized in the prior art.

EP 317878 suggests coating an active compound of this type with a polymeric protective coating, or mixing the compound with a physiologically tolerated buffer which ensures that a pH in the weakly acid to weakly alkaline range is set up in a pharmaceutical formulation in the presence of moisture, or both.

The Dictionnaire Vidal (1985) Cahier Complementaire (p. 10, left col.) describes an enalapril maleate drug named RENITEC which as excipients contains lactose and sodium hydrogen carbonate.

EP 280999B1 suggests similar compositions using alkali or alkaline earth metal carbonates and saccharides as stabilizers for these compounds. Comparative examples therein (see, e.g. Example D) test a formulation with 5.4 mg quinapril hydrochloride, 88.4 mg magnesium carbonate, 5.2 mg gelatin and 1.0 mg magnesium stearate. The formulation, however, shows substantial degradation as compared with lactose-stabilized formulations and is therefore not useful as a practical pharmaceutical formulation.

Said ACE inhibitor compounds have varied sensitivity and specific formulations need to be tested and optimized for different compounds. Consequently, alternative solutions providing stable formulations will be appreciated.

It has now been surprisingly discovered that useful, stable formulations can be produced with use of excipients comprising a basic compound, preferably an alkali or alkaline-earth metal carbonate, and an insoluble alkaline-earth metal hydrogen phosphate is further used as a preferred filler substance. Surprisingly, a saccharide compound for stabilization is not needed in such formulations.

The pH of the formulations of the present invention is dominated by the basic stabilizer.

Tablets of such formulations have good storage stability, dissolution characteristics, and the formulations are suitable for use in drug combinations.

In one embodiment, 45% of magnesium carbonate is mixed with 3.9% quinapril hydrochloride with the addition of 33% calcium hydrogen phosphate (CaHPO₄). Additional specific embodiments and experimental stability test results are disclosed in the Detailed description below and enclosed Examples.

### DETAILED DESCRIPTION

The pharmaceutical formulation of the present invention comprises 0.5-50 wt %, such as about 1-25 wt %, including about 1-15 wt % of a compound of formula I: 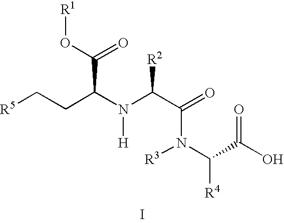
wherein R¹ is hydrogen or alkyl having one to five carbon atoms; R² is hydrogen or C₁-C₄ alkyl or the group 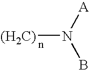
in which A and B independently denote hydrogen or C₁-C₄ alkyl and n is 1-4; R³ and R⁴ together with the atoms they are connected to form a heterocyclic, mono-, di-, or tricyclic ring system which is optionally substituted with C₁-C₄ alkoxy; R⁵ is methyl or phenyl; or any pharmaceutically acceptable salt thereof; 5-90 wt % of an alkali or alkaline earth metal carbonate, such as in the range of about 10-90 wt %, including the range of about 15-75 wt % and preferably in the range of about 25-75 wt %, such as about 25-50 wt % or about 30-50 wt %; 5-90 wt % including the range of about 10-90 wt % of an alkali or alkaline earth metal salt of hydrogen phosphate which is preferably an insoluble alkaline-earth metal salt of hydrogen phosphate, such as in the range of about 15-75 wt %, such as the range of about 20-60 wt %, and preferably in the range of about 25-50 wt %, or in the range of 15-30 wt %;
with the provisio that the formulation does not contain a substantial amount of a saccharide compound.

In this context a substantial amount of a saccharide compound is meant to include any amount that would generally be considered to have a stabilizing effect on the active compound, such as more than about 10 wt %, and more preferably including an amount which is more than about 5 wt %, or even more preferably including any amount of a saccharide compound which is more than about 2 wt %.

The alkaline earth metal carbonate may suitably be selected from magnesium carbonate, sodium hydrogen carbonate and sodium carbonate.

In preferred embodiments, the amount of the alkaline earth metal carbonate is at least the equivalent of the amount of the active compound of formula 1, such as e.g. at least about twice the equivalent, or at least about three times the equivalent of the amount of the active compound.

The term equivalent in this context refers to the conventional ionic equivalent term, one equivalent of a substance participating in a neutralization reaction is that amount of a substance that either contributes or consumes 1 mol of hydrogen ions in that reaction. I.e. for a monoacidic compound such as ramipril and a monobasic alkaline compound such as NaHCO₃, the equivalent ratio of the compounds is the same as the molar ratio; for a diacidic compound such as quinapril HCl stabilized with NaHCO₃, one equivalent of NaHCO₃ equals two moles of NaHCO₃.; and likewise for a diacidic compound and dibasic alikaline compound such as Na₂CO₃, the equivalent ratio is again the same as the molar ratio. (See, e.g. Skoog, West, Holler Fundamentals of Analytical Chemistry 5th Ed., Saunders College Publishing, NY, 1988).

As mentioned, the pH of the formulations are dominated by the basic stabilizing excipient, i.e., the alkali or alkaline-earth metal carbonate.

The ACE inhibitor compound is generally selected from enalapril, delaprii, lisinopril, moexipril, perindopril, quinapril, ramipril, trandolapril, and pharmaceutically acceptable salts thereof. In particular, stable formulations of quinapril or a salt thereof are suitably manufactured according to the invention disclosed herein.

In yet further useful embodiments, the formulation of of the present invention further comprises in the range of about 0.5-50 wt % of a pharmaceutically active compound selected from the group containing diuretics including hydrochlorothiazide; antitussives including dextromethorphan, dextromethorphan hydrobromide, noscapine, carbetapentane citrate, and chlophedianol hydrochloride; antihistamines including chloepheniramine maleate, phenindamine tartrate, pyrilamine maleate, doxylamine succinate, and phenyltoloxamine citrate; decongestants including phenylephedrine hydrochloride, phenylpropanolamine hydrochloride, pseudoephedrine hydrochloride, ephedrine; and alkaloids such as codeine phosphate, codeine sulfate, and morphine. The suitable amount of a further pharmaceutically active compound such as the above listed depends on the particular compound, i.e. the activity of the compound and its suitable dose, and the dose weight of the pharmaceutical formulation.

### EXAMPLES

### Example 1

The following materials were combined by the wet granulation method for the manufacture of 5 mg quinapril tablets.

| | | |
|---|---|---|
| Quinapril hydrochloride | 5.4 | mg |
| Magnesium carbonate | 63 | mg |
| Calcium hydrogen phosphate anhydrous | 46.4 | mg |
| Starch pregelatinized | 21 | mg |
| Croscarmellose sodium | 2.8 | mg |
| Magnesium stearate | 1.4 | mg |

### Example 2

The following materials were processed by wet granulation for 10/12.5 mg tablets with Quinapril and Hydrochlorothiazide.

| | | |
|---|---|---|
| Quinapril hydrochloride | 10.8 | mg |
| Hydrochlorothiazide | 12.5 | mg |
| Magnesium carbonate | 49 | mg |
| Calcium hydrogen phosphate anhydrous | 42.5 | mg |
| Starch pregelatinized | 21 | mg |
| Croscarmellose sodium | 2.8 | mg |
| Magnesium stearate | 1.4 | mg |

### Example 3

Stability of the tablets prepared in the Examples 1-2 were tested at 40° C. for 9 days.

Degradation products (%)

| | Quinapril DKP | Quinaprilat |
|---|---|---|
| Example 1 | 0.2 | 1.2 |
| Example 2 | 0.3 | 0.6 |

(DKP = diketopiperazine)

1. a pharmaceutical formulation comprising:
a. 0.5-50 wt % of a compound of formula I; 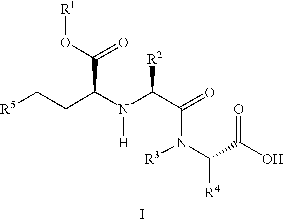
wherein R¹ is hydrogen or alkyl having one to five carbon atoms; R² is hydrogen or C₁-C₄ alkyl or the group 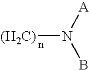
in which A and B independently denote hydrogen or C₁-C₄ alkyl and n is 1-4; R³ and R⁴ together with the atoms they are connected to form a heterocyclic, mono-, di-, or tricyclic ring system which is optionally substituted with C₁-C₄ alkoxy; R⁵ is methyl or phenyl; and pharmaceutically acceptable salts thereof;
b. 5-90 wt % of an alkali or alkaline earth metal carbonate;
c. 5-90 wt % of an insoluble alkaline-earth metal salt of hydrogen phosphate;
with the provisio that the formulation does not contain a substantial amount of a saccharide compound.

2. The formulation of claim 1, wherein the alkali or alkaline-earth metal carbonate is selected from magnesium carbonate, sodium hydrogen carbonate and sodium carbonate.

3. The formulation of claim 1, wherein the amount of the alkaline earth metal carbonate is at least the equivalent of the amount of the active compound of formula I.

4. The formulation of claim 1, wherein the compound of formula I is selected from the group containing enalapril, delapril, lisinopril, moexipril, perindopril, quinapril, ramipril, trandolapril, and pharmaceutically acceptable salts thereof.

5. The formulation of claim 1 wherein the formulation comprises in the range of about 1-15 wt % of the compound of formula 1.

6. The formulation of claim 1 wherein the formulation comprises in the range of about 25-75 wt % of the alkali or alkaline earth metal carbonate.

7. The formulation of claim 6 wherein the formulation comprises in the range of about 30-50 wt % of the alkali or alkaline earth metal carbonate.

8. The formulation of claim 1 wherein the formulation comprises in the range of about 15-75 wt % of the salt of an insoluble alkaline earth metal hydrogen phosphate.

9. The formulation of claim 1 wherein the formulation comprises in the range of about 25-50 wt % of the salt of an insoluble alkaline earth metal hydrogen phosphate.

10. The formulation of claim 4 wherein the compound of formula I is quinapril or a pharmaceutically acceptable salt thereof.

11. The formulation of claim 1 further comprising 0.5-50 wt % of a pharmaceutically active compound selected from the group containing diuretics including hydrochlorothiazide; antitussives including dextromethorphan, dextromethorphan hydrobromide, noscapine, carbetapentane citrate, and chlophedianol hydrochloride; antihistamines including chloepheniramine maleate, phenindamine tartrate, pyrilamine maleate, doxylamine succinate, and phenyltoloxamine citrate; decongestants including phenylephedrine hydrochloride, phenylpropanolamine hydrochloride, pseudoephedrine hydrochloride, ephedrine; and alkaloids such as codeine phosphate, codeine sulfate, and morphine.
